(19) United States
(12) Patent Application Publication
(10) Pub. No.: US 2022/0152127 A1
(43) Pub. Date: May 19, 2022

(54) ENGINEERED LACTOCOCCUS

(71) Applicant: UNIVERSITA DEGLI STUDI DI ROMA "LA SAPIENZA", Roma (RM) (IT)

(72) Inventors: Cinzia MARCHESE, Roma (IT); Antonio ANGELONI, Roma (IT); Simona CECCARELLI, Roma (IT); Sirio D'AMICI, Roma (IT)

(73) Assignee: UNIVERSITA DEGLI STUDI DI ROMA "LA SAPIENZA", Roma (RM) (IT)

(21) Appl. No.: 17/435,588

(22) PCT Filed: Mar. 4, 2020
(86) PCT No.: PCT/EP2020/055705

§ 371 Date: Sep. 1, 2021

(30) Foreign Application Priority Data
Mar. 4, 2019 (IT) 102019000003115

(51) Int. Cl. A61K 35/744; A61K 9/00; C12N 15/74

(52) U.S. Cl. CPC A61K 35/744; A23L 33/135; C12N 15/746; A61K 9/0034

(57) ABSTRACT

The present invention refers to a microorganism characterized in that it is genetically modified to express the human growth factor of keratinocytes (KGF/FGF7) or its functional orthologues, derivatives or fragments.

### FIELD OF THE INVENTION

The present invention relates to a microorganism characterized in that it is genetically modified to express the human growth factor of keratinocytes (KGF/FGF7) or its functional orthologues, derivatives and fragments. The present invention finds its primary application in the medical field, in the treatment of vaginal atrophy conditions induced by menopause.

### PRIOR ART

Vaginal atrophy is a frequent problem in postmenopausal women. In fact, this physiological event causes alterations in the vaginal epithelium, characterized by a thinning of the tissue and reduced lubrication, with consequent inflammation of the vaginal mucosa and the final tract of the urinary tract, which occur in over 50% of menopausal women [Pandit and Ouslander, 1997].

Vaginal atrophy can also occur in premenopausal women, for example after surgical removal of an endometrial carcinoma, or after para-aortic pelvic lymphadenectomy or adjuvant vaginal brachytherapy (VBT), interventions performed to reduce the risk of local recurrence and improve survival of cancer patients [Keys et al., 2004].

The lack of estrogens related to menopause induces a progressive vulvar atrophy, characterized by a thinning of the mucosa, with a 30-50% reduction in the levels of vascularization. The treatment of choice is oral hormone replacement therapy (HRT), which however is not recommended in women previously suffering from oncological diseases [Edey et al., 2018]. Furthermore, the use of HRT presents problems also for healthy women, being related to a higher incidence of cancer [Kim et al., 2018].

The keratinocyte growth factor (KGF/FGF7) (NCBI Reference Sequence: Ncbi accession number NG 029159, GenBank amino acid sequence: Accession number AAB21431.1), a member of the fibroblast growth factor (FGF) family, plays a key role in regulating cell proliferation, migration and differentiation during development and in response to tissue repair and injury processes [Finch and Rubin, 2004]. KGF acts by binding to its specific receptor tyrosine-kinase, FGFR2-IIIb/KGFR, generated through an alternative splicing of the FGFR2 gene and mainly expressed on epithelial cells of different organs, in which it plays a key role in the control of epithelial growth and differentiation [Eswarakumar et al., 2005]. The KGF/KGFR pathway is essential for maintaining the integrity and functionality of epithelial tissues in adults, due to the cytoprotective and regenerative activity of KGF. Indeed, the expression of KGF is strongly upregulated following lesions in various epithelial tissues such as skin, kidneys, bladder, pancreas, stomach and intestine [Werner et al., 1992; Marchese et al., 1995; Brauchle et al., 1996; Werner, 1998]. Furthermore, KGF protects the pulmonary epithelium from lesions and improves distal airway repair by stimulating cell proliferation, inhibiting apoptosis and the formation of oxygen free radicals, and mobilizing progenitor epithelial cells [Gomperts et al., 2007]. It has been shown that treatment with recombinant KGF (Palifermina) protects epithelial cells from a variety of lesions, including radiation-induced damage. Therefore, the pharmacological use of KGF has been approved by the FDA for the treatment of severe oral mucositis resulting from radiotherapy or chemotherapy in patients with haematological tumours or head and neck cancers [Spielberger et al., 2004; Beaven and Shea, 2007; Brizel et al., 2008; Barasch et al., 2009]. On the other hand, the vaginal administration of KGF during the neonatal period in murine models determines an oestrogen-independent proliferation in the vaginal epithelium, suggesting a potential link between oestrogen treatment and activation of KGF/KGFR signalling [Masui et al., 2004]. In this context, the present inventors have previously shown that KGF can be used as an alternative treatment to local administration of estrogens (see International patent application WO2014/023773). The previous patent application refers to the use of KGF and its related pharmaceutical compositions in the treatment of vaginal atrophy, dysuria, vaginal pain and/or vaginal dryness induced by a post-menopausal state, by surgery, by a disease and/or by chemotherapy or radiotherapy. Furthermore, in vitro and in vivo results on the local effects of KGF on vaginal epithelium have been published in an international journal [Ceccarelli et al. 2014]. In particular, the effect of local administration of KGF on vaginal atrophy has been evaluated in vivo on an animal model, represented by female CD1 strain ovariectomized mice, using Pluronic F127, a synthetic amphiphilic polymer, as a vehicle to allow the gradual release of KGF. Kinetic studies showed that the release of KGF was almost complete within 24 hours, thus indicating the need for daily treatment.

The administration of therapeutic molecules directly at the mucosa level due to infections or diseases involving that tissue not only increases their effectiveness and specificity, but also contributes to reducing the side effects compared to systemic routes of administration [Neutra and Kozlowski, 2006; Davis, 2001; Holmgren and Czerkinsky, 2005].

The strategy of administration at the level of the mucous membranes is considered better than the systemic one because of easier execution, it does not require the use of needles and syringes and therefore of trained personnel; furthermore, the immunogenicity of soluble proteins is lower when these are administered through the mucous tissue. Probiotics are vital microorganisms that exert beneficial effects on the host when provided in adequate quantities. Probiotics are generally isolated from stool samples from normal individuals, mostly from breastfed babies. Examples of probiotics for human use are those belonging to the Lactobacillus (e.g. L. acidophilus, L. casei), Bifidobacterium (e.g. B. animalis subsp lactis), or Saccharomyces (e.g. S. boulardii, S. florentinus) species. Lactococcus Lactis is an optional non-invasive and mesophilic heterofermentative bacterium (ideal growth temperature around 30° C.), widely used in the dairy industry [Pontes et al., 2011]. It belongs to the family of lactic acid bacteria (LAB), which represents a heterogeneous group of gram-positive microorganisms of great technological importance [del Carmen et al., 2011]. LABs play a key role in maintaining the balance of intestinal and vaginal microflora, and can be isolated from vaginal secretions of healthy women [Fuller, 1989; Reid and Bocking, 2003; Hoesl and Altwein 2005; Todorov et al., 2007]. Thanks to their numerous beneficial properties, and their status as generally recognized as safe (GRAS) organisms, LABs are the most commonly used probiotic microorganisms, and can be defined as “live microorganisms which, when administered in adequate quantities, confer benefits to the host” [FAO/WHO, 2001]. Currently, L. lactis is the best described member of the LAB and is considered the model organism of this group, not only for its economic importance, but also for the following characteristics:
has a fully sequenced genome [Bolotin et al., 2009];
it is genetically easy to manipulate;
many genetic tools have already been developed for this species [de Vos, 1999].

The use of engineered lactobacilli to produce molecules of interest directly in situ is already known.

For example, patent application EP3067058 refers to a method for producing therapeutic cannabinoids characterized by the administration to a host of the subspecies of Lactobacillus Paracasei, subspecies Paracasei F19 probiotic, genetically modified to produce and secrete cannabinoids of Cannabis sativa, in association with the caproic acid, able to establish the enzymatic reactions of the biosynthetic pathway that leads to the production of cannabinoids directly in situ in said host.

Application WO2014066945 refers to genetically modified probiotics expressing recombinant phenylalanine ammonia lyase (PAL) to treat phenylketonuria.

L. lactis can be genetically engineered to efficiently produce and secrete several proteins, a feature recently used by scientists to convey therapeutic proteins directly to mucous membranes, particularly through intranasal, oral or genital mucosal surfaces.

Sufficient data are currently available to support the use of recombinant LABs, in particular L. lactis, to convey therapeutic proteins to mucosal tissues [Bermúdez-Humarán et al., 2004]. Therefore, there is still the need to overcome the above described disadvantages and to improve both the effectiveness of the KGF-based therapy for the treatment of vaginal atrophy and the compliance of patients.

In particular, there is a need to obtain an engineered microorganism capable of directly colonizing the vaginal mucosa, being controllable in the production of KGF by a safe inducible system in patients.

### SUMMARY OF THE INVENTION

For the above described drawbacks and to improve both the effectiveness of the KGF-based therapy for the treatment of vaginal atrophy and the “compliance” of the patients, the present inventors applied a strategy based on the creation of a genetically modified strain for the KGF production. The present authors used a strain of L. lactis, defined as a GRAS (Generally Recognized As Safe) organism, previously used as a vehicle for various biomedical products, such as vaccines, antigens and other therapeutic agents [Bron and Kleerebezem, 2018] to produce recombinant KGF.

Therefore, the present inventors have genetically modified a strain of L. lactis to make it able to produce KGF in a controlled manner, using a controlled expression system using Nisin (NICE system), and inserting a secretion signal peptide upstream of the KGF gene specific for L. lactis, Usp45. The engineered lactobacillus object of the present invention is able to directly colonize the vaginal mucosa, and its production of KGF can be controlled by the NICE inducible system, through the oral intake of Nisin. Furthermore, the proliferative activity of recombinant KGF produced by L. lactis on primary cells of vaginal epithelium is demonstrated here, thus supporting the therapeutic activity of the microorganism of the invention.

### DETAILED DESCRIPTION OF THE INVENTION

Therefore, it is an object of the present invention a microorganism characterized in that it is genetically modified to express the human growth factor of keratinocytes (KGF/FGF7) or its functional orthologues, derivatives or fragments. Preferably the microorganism is a probiotic. More preferably the microorganism is a GRAS organism.

Preferably, said microorganism or probiotic is a lactic acid bacterium, more preferably of the genus Lactobacillus or Lactococcus.

Preferably, said microorganism belongs to the genus Lactococcus, preferably Lactococcus Lactis. More preferably said Lactococcus Lactis is the NZ3900 strain.

In a preferred embodiment, the microorganism as defined above has been genetically modified with a recombinant polynucleotide comprising a nucleic acid encoding the KGF, its functional orthologues, derivatives or functional fragments and/or said microorganism comprises a plasmid comprising a nucleic acid coding for the human keratinocyte growth factor (KGF/FGF7) or its functional orthologues, derivatives or fragments.

Said nucleic acid encoding the human KGF, its functional orthologues, derivatives or fragments is preferably operatively linked to an expression promoter, preferably inducible, more preferably a nisin-inducible promoter, e.g. the PnisA promoter.

Preferably, said KGF, its functional orthologues, derivatives or fragments are secreted.

Preferably, said KGF, its orthologues, derivatives or functional fragments are expressed as a fusion protein with a secretion signal that works in the microorganism, such as the probiotic, more preferably said signal is Usp45 or PrtP.

Preferably the Usp45 signal has a sequence having at least 80% identity with the SEQ ID NO: 6 or 7. The plasmid described above therefore in a preferred embodiment comprises a sequence having at least 80% identity with the SEQ ID NO: 6.

Preferably the PrtP signal has a sequence having at least 80% identity with the SEQ ID NO: 10. The plasmid described above therefore in a preferred embodiment comprises a sequence coding for a sequence having at least 80% identity with the SEQ ID NO: 10.

Preferably said KGF comprises a sequence having at least 80% identity with SEQ ID NO:2, 3 or with the sequence aa. 56-194 of SEQ ID NO:3.

The nucleic acid encoding the KGF comprises preferably a sequence having at least 80% identity with the SEQ ID NO: 1 or 11.

The plasmid described above therefore in a preferred embodiment comprises a sequence having at least 80% identity with the SEQ ID NO: 1 or 11 or with the sequences encoding the SEQ ID NO: 2, 3 or the sequence aa. 56-194 of SEQ ID NO:3.

Preferably, the microorganism according to the invention is able to colonize the vaginal mucosa. Preferably said microorganism releases in a controlled manner the KGF. The production of KGF can be controlled by an inducible system, e.g. NICE system, through the oral intake of Nisin. The KGF produced by the microorganism of the present invention has effects on primary cells of vaginal epithelium.

Further objects of the invention are a composition comprising said microorganism and at least one excipient and a pharmaceutical composition comprising said microorganism and at least one pharmaceutically acceptable excipient.

The microorganism may be or not in a lyophilized form. Preferably said microorganism is in an amount of 12-24×10¹¹-12-24×10¹² cfu per gram of composition.

Another object of the invention is a combination which comprises:
a) the microorganism as above defined or the composition as above defined and
b) an inducer of the expression promoter.

The microorganism or the composition or the combination according to the invention are preferably for medical use, more preferably for use in the treatment of vaginal atrophy, dysuria, vaginal pain and/or vaginal drying induced by a post-menopausal status, by surgery, by a pathology and/or by chemotherapy or radiotherapy.

The microorganism or the composition or the combination according to the invention are preferably for use in the production of human KGF or its functional orthologues, derivatives or fragments directly in situ in a host, more preferably in the human vaginal mucosa. Preferably the microorganism is administered topically on the vagina mucosa, preferably by introduction in the vaginal cavity, for example by hydrogels, vaginal tablets, suppositories, particulate systems and intravaginal rings. Even more preferably the microorganism has been genetically modified with a recombinant polynucleotide comprising a nucleic acid encoding the human KGF, its functional orthologues, derivatives or functional fragments and/or said microorganism comprises a plasmid comprising a nucleic acid coding for the human keratinocyte growth factor (KGF/FGF7) or its functional orthologues, derivatives or fragments. More preferably the nucleic acid encoding the human KGF, its functional orthologues, derivatives or fragments is operatively linked to an inducible expression promoter.

The inducible expression promoter is preferably a nisin-inducible promoter, preferably the PnisA promoter.

Preferably, an inducer of the expression promoter, preferably nisin, is also administered. More preferably the promoter is the PnisA promoter. Preferably the expression promoter inducer is administered orally.

A further object of the present invention is a combination which comprises the composition as defined above and an inducer of the expression promoter, where the composition can be administered on the vaginal mucosa for example by hydrogels, vaginal tablets, suppositories, particulate systems and intravaginal rings, while the expression promoter inducer can be administered orally.

For “Keratinocyte growth factor” or “KGF/FGF7” or “KGF” it is intended the entire wild type protein KGF (NCBI Reference Sequence: NP 002000.1; GenBank amino acid sequence: CAG46799.1):

```
(SEQ ID No: 3)
    1 mhkwiltwil ptllyrscfh iiclvgtisl
        acndmtpeqm atnvncsspe rhtrsydyme
  61 ggdirvrrlf crtqwylrid krgkvkgtqe
        mknnynimei rtvavgivai kgvesefyla
121 mnkegklyak kecnedcnfk elilenhynt
        yasakwthng gemfvalnqk gipvrgkktk
181 keqktahflp mait
```

or the amino acid sequence:

```
(SEQ ID NO: 2)
CNDMTPEQMATNVNCSSPERHTRSYDYMEGGDIRV
RRLFCRTQWYLRIDKRGKVKGTQEMKNNYNIMEIR
TVAVGIVAIKGVESEFYLAMNKEGKLYAKKECNED
CNFKELILENHYNTYASAKWTHNGGEMFVALNQKG
IPVRGKKTKKEQKTAHFLPMAIT
or the nucleotide sequence
(NCBI reference Sequence: NM 002009.3
(SEQ ID No. 11):
AGTTTTAATTGCTTCCAATGAGGTCAGCAAAGGTA
TTTATCGAAAAGCCCTGAATAAAAGGCTCACACAC
ACACACAAGCACACACGCGCTCACACACAGAGAGA
AAATCCTTCTGCCTGTTGATTTATGGAAACAATTA
TGATTCTGCTGGAGAACTTTTCAGCTGAGAAATAG
TTTGTAGCTACAGTAGAAAGGCTCAAGTTGCACCA
GGCAGACAACAGACATGGAATTCTTATATATCCAG
CTGTTAGCAACAAAACAAAAGTCAAATAGCAAACA
GCGTCACAGCAACTGAACTTACTACGAACTGTTTT
TATGAGGATTTATCAACAGAGTTATTTAAGGAGGA
ATCCTGTGTTGTTATCAGGAACTAAAAGGATAAGG
CTAACAATTTGGAAAGAGCAACTACTCTTTCTTAA
ATCAATCTACAATTCACAGATAGGAAGAGGTCAAT
GACCTAGGAGTAACAATCAACTCAAGATTCATTTT
CATTATGTTATTCATGAACACCCGGAGCACTACAC
TATAATGCACAAATGGATACTGACATGGATCCTGC
CAACTTTGCTCTACAGATCATGCTTTCACATTATC
TGTCTAGTGGGTACTATATCTTTAGCTTGCAATGA
CATGACTCCAGAGCAAATGGCTACAAATGTGAACT
GTTCCAGCCCTGAGCGACACACAAGAAGTTATGAT
TACATGGAAGGAGGGGATATAAGAGTGAGAAGACT
CTTCTGTCGAACACAGTGGTACCTGAGGATCGATA
AAAGAGGCAAAGTAAAAGGGACCCAAGAGATGAAG
AATAATTACAATATCATGGAAATCAGGACAGTGGC
AGTTGGAATTGTGGCAATCAAAGGGGTGGAAAGTG
AATTCTATCTTGCAATGAACAAGGAAGGAAAACTC
TATGCAAAGAAAGAATGCAATGAAGATTGTAACTT
CAAAGAACTAATTCTGGAAAACCATTACAACACAT
ATGCATCAGCTAAATGGACACACAACGGAGGGGAA
ATGTTTGTTGCCTTAAATCAAAAGGGGATTCCTGT
AAGAGGAAAAAAAACGAAGAAAGAACAAAAAACAG
CCCACTTTCTTCCTATGGCAATAACTTAATTGCAT
ATGGTATATAAAGAACCAGTTCCAGCAGGGAGATT
TCTTTAAGTGGACTGTTTTCTTTCTTCTCAAAATT
TTCTTTCCTTTTATTTTTTAGTAATCAAGAAAGGC
TGGAAAACTACTGAAAAACTGATCAAGCTGGACTT
GTGCATTTATGTTTGTTTTAAGACACTGCATTAAA
GAAAGATTTGAAAAGTATACACAAAAATCAGATTT
AGTAACTAAAGGTTGTAAAAAATTGTAAAACTGGT
TGTACAATCATGATGTTAGTAACAGTAATTTTTTT
CTTAAATTAATTTACCCTTAAGAGTATGTTAGATT
TGATTATCTGATAATGATTATTTAAATATTCCTAT
CTGCTTATAAAATGGCTGCTATAATAATAATAATA
CAGATGTTGTTATATAAGGTATATCAGACCTACAG
GCTTCTGGCAGGATTTGTCAGATAATCAAGCCACA
CTAACTATGGAAAATGAGCAGCATTTTAAATGCTT
TCTAGTGAAAAATTATAATCTACTTAAACTCTAAT
CAGAAAAAAAATTCTCAAAAAAACTATTATGAAAG
TCAATAAAATAGATAATTTAACAAAAGTACAGGAT
TAGAACATGCTTATACCTATAAATAAGAACAAAAT
TTCTAATGCTGCTCAAGTGGAAAGGGTATTGCTAA
AAGGATGTTTCCAAAAATCTTGTATATAAGATAGC
AACAGTGATTGATGATAATACTGTACTTCATCTTA
CTTGCCACAAAATAACATTTTATAAATCCTCAAAG
TAAAATTGAGAAATCTTTAAGTTTTTTTCAAGTAA
CATAATCTATCTTTGTATAATTCATATTTGGGAAT
ATGGCTTTTAATAATGTTCTTCCCACAAATAATCA
TGCTTTTTTCCTATGGTTACAGCATTAAACTCTAT
TTTAAGTTGTTTTTGAACTTTATTGTTTTGTTATT
TAAGTTTATGTTATTTATAAAAAAAAAACCTTAAT
AAGCTGTATCTGTTTCATATGCTTTTAATTTTAAA
GGAATAACAAAACTGTCTGGCTCAACGGCAAGTTT
CCCTCCCTTTTCTGACTGACACTAAGTCTAGCACA
CAGCACTTGGGCCAGCAAATCCTGGAAGGCAGACA
AAAATAAGAGCCTGAAGCAATGCTTACAATAGATG
TCTCACACAGAACAATACAAATATGTAAAAAATCT
TTCACCACATATTCTTGCCAATTAATTGGATCATA
TAAGTAAAATCATTACAAATATAAGTATTTACAGG
ATTTTAAAGTTAGAATATATTTGAATGCATGGGTA
GAAAATATCATATTTTAAAACTATGTATATTTAAA
TTTAGTAATTTTCTAATCTCTAGAAATCTCTGCTG
TTCAAAAGGTGGCAGCACTGAAAGTTGTTTTCCTG
TTAGATGGCAAGAGCACAATGCCCAAAATAGAAGA
TGCAGTTAAGAATAAGGGGCCCTGAATGTCATGAA
GGCTTGAGGTCAGCCTACAGATAACAGGATTATTA
CAAGGATGAATTTCCACTTCAAAAGTCTTTCATTG
GCAGATCTTGGTAGCACTTTATATGTTCACCAATG
GGAGGTCAATATTTATCTAATTTAAAAGGTATGCT
AACCACTGTGGTTTTAATTTCAAAATATTTGTCAT
TCAAGTCCCTTTACATAAATAGTATTTGGTAATAC
ATTTATAGATGAGAGTTATATGAAAAGGCTAGGTC
AACAAAAACAATAGATTCATTTAATTTTCCTGTGG
TTGACCTATACGACCAGGATGTAGAAAACTAGAAA
GAACTGCCCTTCCTCAGATATACTCTTGGGAGAGA
GCATGAATGGTATTCTGAACTATCACCTGATTCAA
GGACTTTGCTAGCTAGGTTTTGAGGTCAGGCTTCA
GTAACTGTAGTCTTGTGAGCATATTGAGGGCAGAG
GAGGACTTAGTTTTTCATATGTGTTTCCTTAGTGC
CTAGCAGACTATCTGTTCATAATCAGTTTTCAGTG
TGAATTCACTGAATGTTTATAGACAAAAGAAAATA
CACACTAAAACTAATCTTCATTTTAAAAGGGTAAA
ACATGACTATACAGAAATTTAAATAGAAATAGTGT
ATATACATATAAAATACAAGCTATGTTAGGACCAA
ATGCTCTTTGTCTATGGAGTTATACTTCCATCAAA
TTACATAGCAATGCTGAATTAGGCAAAACCAACAT
TTAGTGGTAAATCCATTCCTGGTAGTATAAGTCAC
CTAAAAAAGACTTCTAGAAATATGTACTTTAATTA
TTTGTTTTTCTCCTATTTTTAAATTTATTATGCAA
ATTTTAGAAAATAAAATTTGCTCTAGTTACACACC
TTTAGAATTCTAGAATATTAAAACTGTAAGGGGCC
TCCATCCCTCTTACTCATTTGTAGTCTAGGAAATT
GAGATTTTGATACACCTAAGGTCACGCAGCTGGGT
AGATATACAGCTGTCACAAGAGTCTAGATCAGTTA
GCACATGCTTTCTACTCTTCGATTATTAGTATTAT
TAGCTAATGGTCTTTGGCATGTTTTTGTTTTTTAT
TTCTGTTGAGATATAGCCTTTACATTTGTACACAA
ATGTGACTATGTCTTGGCAATGCACTTCATACACA
ATGACTAATCTATACTGTGATGATTTGACTCAAAA
GGAGAAAAGAAATTATGTAGTTTTCAATTCTGATT
CCTATTCACCTTTTGTTTATGAATGGAAAGCTTTG
TGCAAAATATACATATAAGCAGAGTAAGCCTTTTA
AAAATGTTCTTTGAAAGATAAAATTAAATACATGA
GTTTCTAACAATTAGA
or the GenBank gene sequence:
(access number NC 000015.10)
(SEQ ID NO: 1)
TGC AAT GAC ATG ACT CCA GAG CAA ATG GCT ACA
AAT GTG AAC TGT TCC AGC CCT GAG CGA CAC ACA
AGA AGT TAT GAT TAC ATG GAA GGA GGG GAT ATA
AGA GTG AGA AGA CTC TTC TGT CGA ACA CAG TGG
TAC CTG AGG ATC GAT AAA AGA GGC AAA GTA AAA
GGG ACC CAA GAG ATG AAG AAT AAT TAC AAT ATC
ATG GAA ATC AGG ACA GTG GCA GTT GGA ATT GTG
GCA ATC AAA GGG GTG GAA AGT GAA TTC TAT CTT
GCA ATG AAC AAG GAA GGA AAA CTC TAT GCA AAG
AAA GAA TGC AAT GAA GAT TGT AAC TTC AAA GAA
CTA ATT CTG GAA AAC CAT TAC AAC ACA TAT GCA
TCA GCT AAA TGG ACA CAC AAC GGA GGG GAA ATG
TTT GTT GCC TTA AAT CAA AAG GGG ATT CCT GTA
AGA GGA AAA AAA ACG AAG AAA GAA CAA AAA ACA
GCC CAC TTT CTT CCT ATG GCA ATA ACT TAA(stop),
the human recombinant KGF
(SwissProt amino acid
sequence: P21781 # (SEQ ID No. 3):
                10 20 30
MHKWILTWIL PTLLYRSCFH IICLVGTISL
                40 50 60
ACNDMTPSQM ATNVNCSSPE RHTRSYDYME
                70 80 90
GGDIRVRRLF CRTQWYLRID KRGKVKGTQE
              100 110 120
MKNNYNIMEI RTVAVGIVAI KGVESEFYLA
              130 140 150
MNKEGKLYAK KECNEDCNFK ELILENHYNT
              160 170 180
YASAKWTHKG GEMFVALNQK GIPVRGKKTK
              190
KEQKTAHFLP MAIT
or the human recombinant KGF Palifermin
(DrugBank: DB00039 (fragment aa. 56 to aa.
194 of SEQ ID No. 3 corresponding
to SEQ ID NO: 12):
YDYMEGGDIRVRRLFCRTQWYLRIDKRGKVKGTQEMKNNYNIMEI
RTVAVGIVAIKGVESEFYLAMNKEGKLYAKKECNEDCNFKELILE
NHYNTYASAKWTHNGGEMFVALNQKGIPVRGKKTKKEQKTAHFLP
MAIT
```

or functional allelic variants, or orthologues, fragments, mutants, derivatives or analogues thereof.

In the present invention, functional variants, orthologues, fragments, mutants, derivatives or analogues possess the same pharmacological activity as the KGF protein.

The object of the present invention is therefore a microorganism, able to integrate into the bacterial flora of the host, and comprising a plasmid containing the KGF gene.

The microorganism according to the invention is preferably administered in a single administration by vaginal route, preferably in a dose of at least 12×10¹¹ colony forming units (cfu). The inducer of KGF secretion, for example nisin, is preferably administered at least once a day.

The microorganism according to the invention or the composition comprising the same can be administered by hydrogels, vaginal tablets, suppositories, particulate systems and intravaginal rings.

Preferably the present composition and the present microorganism are for use in the treatment of conditions of vaginal atrophy and/or vaginal atrophy related to dysuria and/or vaginal atrophy related to vaginal pain and/or dryness induced by a chemotherapy treatment. Preferably said orthologues, derivatives and fragments possess the same therapeutic properties as KGF. Preferably, the chemotherapeutic agent is tamoxifen or an anticancer drug belonging to the family of selective oestrogen receptor modulators. Preferably, the microorganism according to the invention was previously genetically modified by transformation with a nucleotide vector comprising a nucleic acid bearing the KGF gene, or functional allelic variants, orthologues, fragments, mutants, derivatives or analogues thereof.

Preferably the above described microorganism is authorized for use in food for humans or animals. Preferably the microorganism according to the invention is incapsulated.

Preferably the microorganism or composition for use according to the invention is administered in a dose of 12-24×10¹¹-12-24×10¹² cfu.

Preferably, the microorganism or composition according to the invention are formulated as a medicament or nutraceutical. Preferably, the composition according to the invention is administered by hydrogels, vaginal tablets, suppositories, particulate systems or intravaginal rings. The inducer, such as nisin, will be administered preferably orally. A further object of the invention is a method for producing the microorganism as defined above comprising the genetic modification of at least one Lactococcus Lactis cell by means of a recombinant polynucleotide comprising the KGF to obtain a KGF-expressing microorganism. To engineer L. lactis, the present inventors used the NICE system (nisin-controlled expression system) which was tested and demonstrated its versatility in other LAB, such as Leuconostoc lactis, Lactobacillus helveticus, Streptococcus sp., Bacillus sp., Enterococcus sp. [Eichenbaum et al., 1998] and Lactobacillus plantarum [Pavan et al., 2000]. Nisin is a food preservative and a natural, toxicologically safe antibacterial. It is considered natural because it is a polypeptide produced by some strains of L. lactis for food use [Delves-Broughton et al., 1996]. All the bacteria in which this system was tested showed a dose-response profile for nisin for beta-glucuronidase activity, characterized by a level of undetectable protein activity in non-inducing conditions and an increase in levels (from 10 to 60 times) induced by the external addition of sub-inhibitory quantities of nisin. This feature allowed to exploit the NICE system for the use of recombinant LABs as systems for administering vaccines and biotherapeutics at the level of the intestinal mucosa [Poelvoorde et al., 2007]. The success of the Phase I clinical trial of an interleukin-10 L. lactis secreting strain for the treatment of Crohn's disease has opened new horizons for the use of genetically modified LAB as a therapeutic vehicle [Bermúdez-Humarán, 2009].

In the context of the present invention, when reference is made to specific DNA sequences, it is understood that even the RNA molecules identical to the said polynucleotides, except for the fact that the RNA sequence contains uracil instead of thymine and the skeleton of the RNA molecule contains ribose instead of deoxyribose, RNA sequences complementary to the sequences described therein, functional fragments, mutants and their derivatives, proteins encoded by them, functional fragments, mutants and their derivatives are included in the invention. Also included in the present invention are nucleic acid or amino acid sequences derived from the nucleotide or amino acid sequences shown in the present invention, e.g. functional fragments, mutants, derivatives, analogues and sequences with a % of identity of at least 70% with the above mentioned sequences. The term “complementary” sequence refers to a polynucleotide which is not identical to the sequence but has a basic sequence complementary to the first sequence or encodes the same amino acid sequence of the first sequence. A complementary sequence may include DNA and RNA polynucleotides. The term “functional” can be understood as being able to maintain the same activity. The term “fragment” refers to polynucleotides which preferably have a length of at least 1000 nucleotides, 1100 nucleotides, 1200 nucleotides, 1300 nucleotides, 1400 nucleotides, 1500 nucleotides, etc. or to polypeptides which preferably have a length of at least 10aa, 20aa, 30aa, 40aa, 50 aa, 100 aa, 150 aa, 200 aa, 250 aa, 300 aa., etc. “Derivatives” can be recombinant or synthetic. In the context of the present invention, the term “derivatives” when referred to protein indicates a chemically modified protein or an analogue thereof, where at least one substituent is not present in the unmodified protein or an analogue thereof, i.e. a protein that is covalently modified. Typical modifications are ammuines, carbohydrates, alkyl groups, acyl groups, esters and the like. As used herein, the term “derivatives” also refers to longer or shorter sequences of polynucleotides/proteins and/or having, for example, an identity percentage of at least 70%, 75%, 80%, 85%, 86%, 87%, 88%, 89%, 90%, 91%, 92%, 93%, 94%, 95%, 96%, 97%, 98%, more preferably at least 99%, with the sequences here described. In the present invention, “at least 70% identity” indicates that the identity can be a sequence identity of at least 70%, or 75%, or 80%, or 85%, or 90%, or 95% or 100% compared to the indicated sequences. This applies to all the aforementioned % of identity. Preferably, the % of identity concerns the entire length of the indicated sequence. The alignment finalized to determine the percent amino acid sequence identity can be achieved in various ways that are within the skill in the art knowledge, for instance, using publicly available computer software such as BLAST, BLAST-2, ALIGN or Megalign software (DNASTAR). The present invention finds application both in the treatment of human beings and in the veterinary sector. The derivative of the invention also includes “functional mutants” of the polypeptides, which are polypeptides that can be generated by mutating one or more amino acids in their sequences and that maintains their activity. In fact, the polypeptide of the invention, if required, can be modified in vitro and/or in vivo, for example by glycosylation, myristylation, amidation, carboxylation or phosphorylation, and can be obtained, for example, by known synthetic or recombinant techniques in the art. In the present invention “functional” is intended for example as “maintaining its activity” e.g. immunomodulatory activity or anti-inflammatory activity. Also part of the invention are polynucleotides that have the same nucleotide sequences as a polynucleotide exemplified herein except for nucleotide substitutions, additions or deletions within the polynucleotide sequence, as long as these variant polynucleotides substantially retain the same relevant functional activity as the polynucleotides specifically exemplified herein (for example, they code a protein having the same amino acid sequence or the same functional activity encoded by the exemplified polynucleotide). Therefore, the polynucleotides described herein should be understood to include mutants, derivatives, variants and fragments, as discussed above, of specifically exemplified sequences. The present invention also contemplates those polynucleotide molecules having sequences which are sufficiently homologous with the polynucleotide sequences of the invention so as to allow hybridization with this sequence under stringent standard conditions and standard methods (Maniatis, T. et al, 1982). The polynucleotides described herein can also be defined in terms of more particular identity and/or similarity intervals with those here exemplified. The identity of the sequence typically will be greater than 60%, preferably greater than 75%, more preferably greater than 80%, still more preferably greater than 90% and may be greater than 95%. The identity and/or similarity of a sequence can be of 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 or 99% or higher with respect to a sequence here exemplified. In the context of the present invention, the microorganism as defined above has been genetically modified by transformation. Each transformation method known to the expert in the art can be used, for example by chemical (CaCl₂) or physical (electroporation) methods. The term “plasmid” as used herein refers to any molecule capable of autonomous replication that is suitable for transforming a receiving bacterial strain and containing DNA sequences that direct and/or control the expression of heterologous DNA sequences inserted. Different types of plasmids can be used, such as those with low or high number of copies, expression plasmids and cosmids. The microorganism according to the invention can also be defined as “recombinant”. The present microorganism can colonize the vaginal mucosa thus presenting the advantage of limiting repeated administrations of the microorganism. The inducible KGF expression system inserted in a microorganism which is able to colonize the vaginal mucosa, according to the invention, allows a controlled and prolonged release of KGF, thus ameliorating the therapeutic effect of KGF ad favouring patients compliance. Moreover, the inducer that is administered is preferably a food supplement as nisin.

The present invention will now be depicted with non-limiting examples, with reference to the following figures.

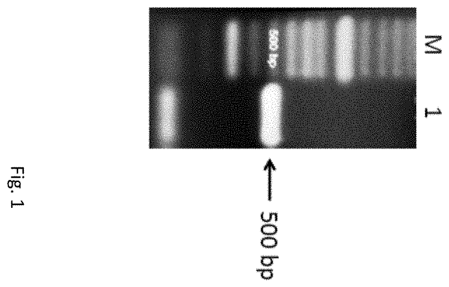

FIG. 1. 1% agarose gel electrophoresis showing a band of 500 bp corresponding to the KGF gene amplified by means of specific primers. Lane M, molecular weight marker of 1 Kb DNA; lane 1, PCR product of KGF gene amplification (500 bp, arrow)

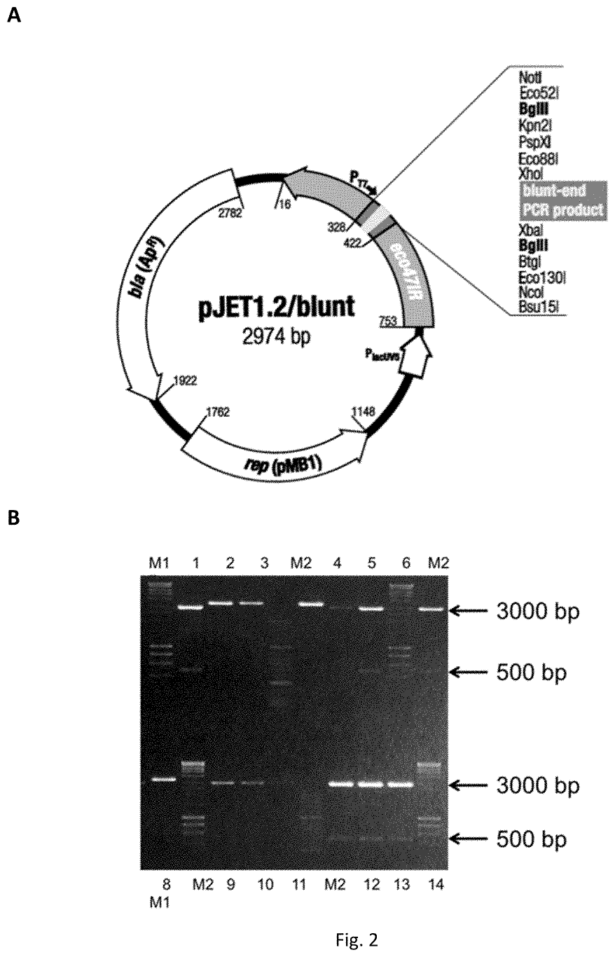

FIG. 2. KGF gene cloning in the vector pJet1.2/blunt. A. Map of the vector pJet1.2/blunt. B. double digestion of the recombinant vector with the restriction enzymes HindIII and EcoRI: Lanes M1, molecular weight marker 1-kb DNA; lanes M2, molecular weight marker 50-bp DNA; lanes 1-14, recombinant digested plasmids. The arrows indicate the expected bands in the KGF-positive clones (3000 bp and 500 bp)

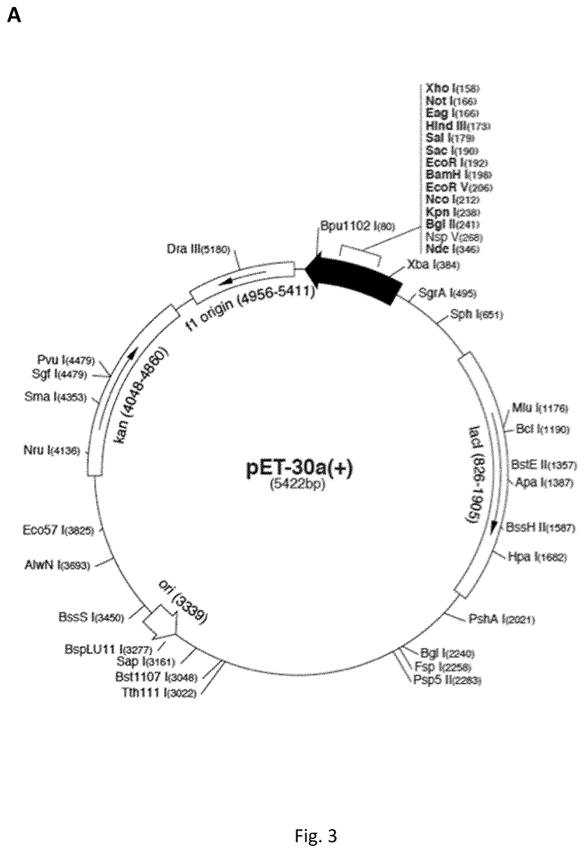

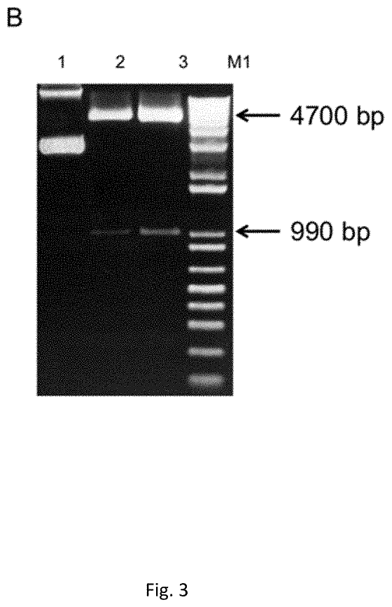

FIG. 3. Cloning of the KGF gene in the vector pET30a. A. Map of the vector pET30a. B. double digestion of the recombinant vector with the restriction enzymes SphI and XhoI: Lane 1, undigested recombinant plasmid; lanes 2 and 3, digested recombinant plasmids; lane M1, 1-kb DNA molecular weight marker. The arrows indicate the expected bands in the KGF-positive clones (4700 bp and 990 bp)

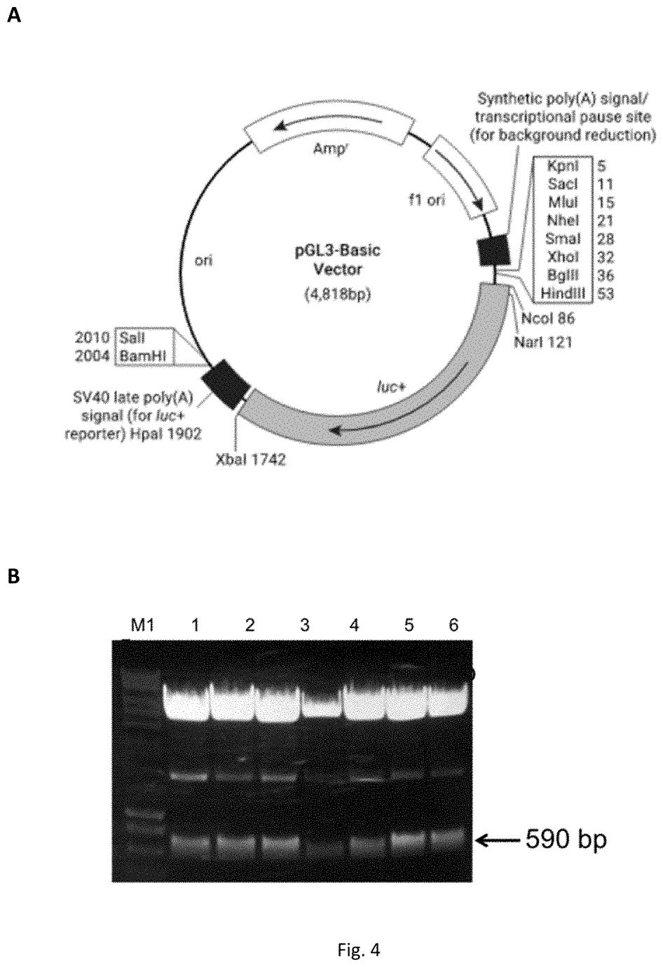

FIG. 4. Cloning of the Usp45-KGF construct in the pGL3-basic vector. A. Map of the pGL3-basic vector. B. double digestion of the recombinant vector with the restriction enzymes HindIII and BglII: Lane M1, 1-kb DNA molecular weight marker; lanes 1-7, recombinant digested plasmids. The arrows indicate the presence of the Usp45-KGF insert in positive clones

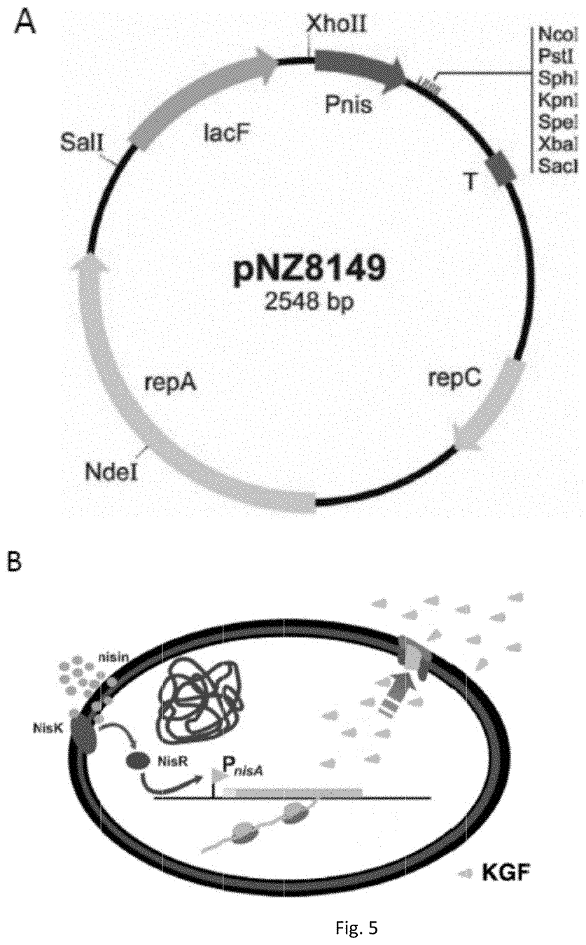

FIG. 5. KGF gene expression system under the control of Ni sin (NICE) for L. lactis. A. Map of the vector pZN8149. B. Schematic representation of the NICE system for the controlled expression of KGF. Gene expression in response to Nisin stimulus involves a sensor protein (NisK), located in the plasma membrane, and a cytoplasmic response regulator (NisR), which controls the transcriptional activation of the promoter (PnisA). Thanks to the presence of the Usp45 secretion signal, after induction with Nisin the KGF protein is produced and secreted in the culture medium

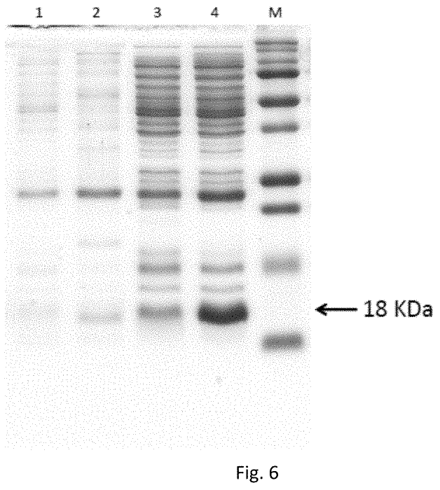

FIG. 6. Analysis of KGF protein expression at various induction times with Nisin. Cell lysates of L. Lactis, strain NZ3900, transformed with the vector pNZKGF, before and after induction with Nisin. Lane 1, cells not induced; lanes 2-4, cells induced with 0.5 ng/ml of Nisin for 3, 12 or 24 h respectively; lane M, molecular weight marker. The arrow indicates the band corresponding to the molecular weight of the KGF protein (18 KDa)

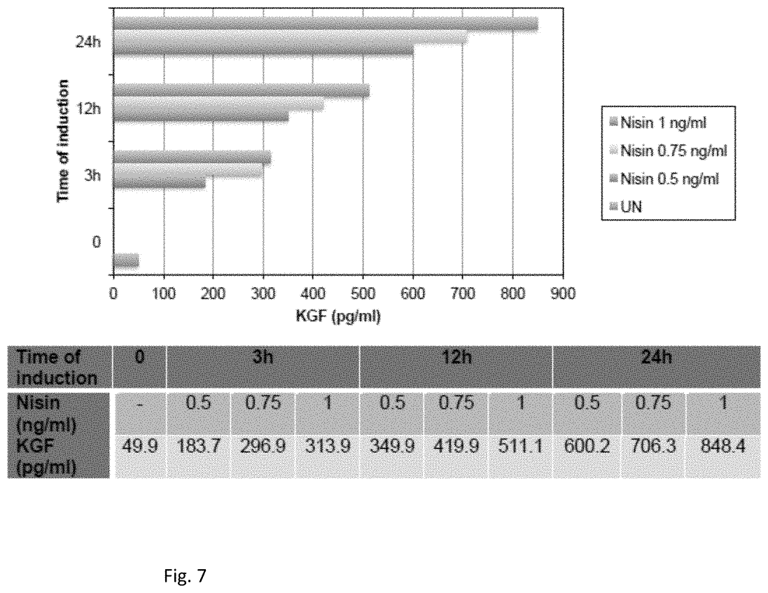

FIG. 7. Analysis of KGF secretion at various induction times and at various concentrations of Nisin. KGF concentration in the supernatants of L. Lactis cells transformed with the pNZKGF vector, induced or induced with 0.5, 0.75 or 1 ng/ml of Nisin for 3, 12 or 24 h, measured by KGF specific ELISA kit

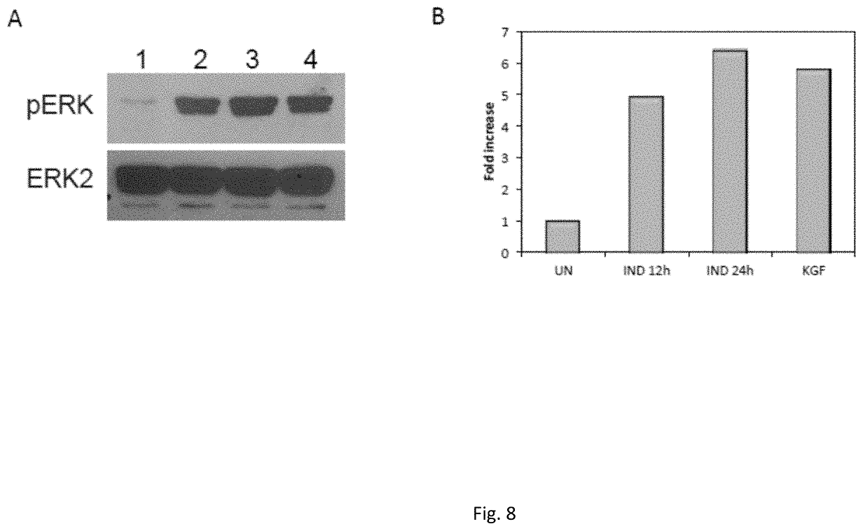

FIG. 8. Analysis of ERK pathway activation by KGF produced by L. lactis. A. Western blot analysis of ERK phosphorylation in HVM cells (human vaginal mucosa) after treatment with L. lactis supernatants transformed with the pNZKGF vector and induced with Nisin. Human recombined KGF was used as a positive control. ERK2 was used as a load control. Lane 1, supernatant not induced; lane 2, induced supernatant for 12 h; lane 3, induced supernatant for 24 h; lane 4, KGF. B. Densitometric analysis of the bands, reported in graph as relative expression with respect to the not induced supernatant (NT)

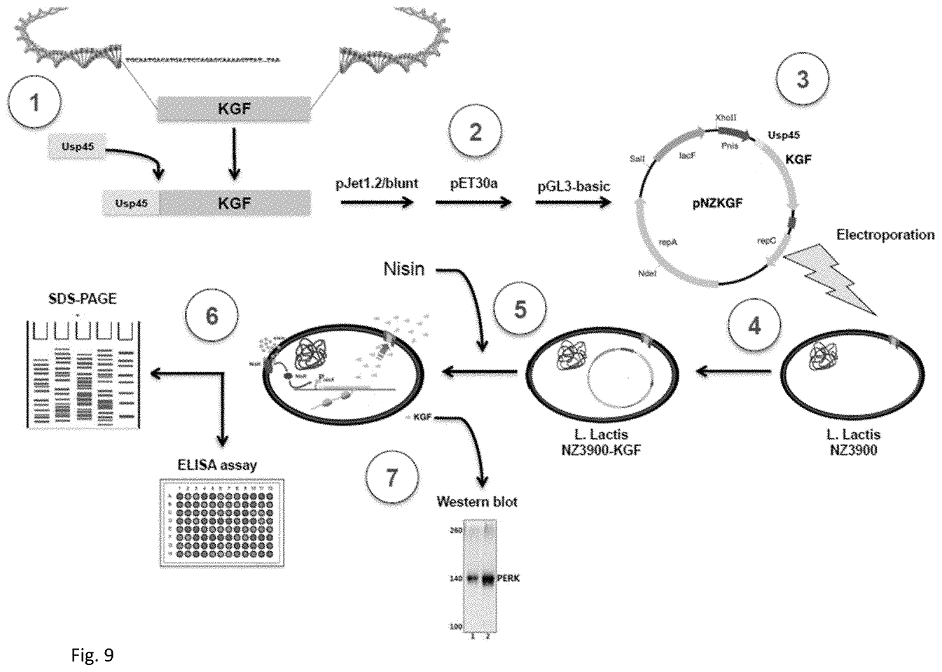

FIG. 9. Schematic representation of the generation of a KGF-producing L. lactis strain.

### EXAMPLE

Materials and Methods

KGF Gene Amplification

The DNA sequence encoding the KGF was obtained from GenBank (access number NC_000015.10).

KGF Gene Sequence:

(SEQ ID NO: 1)
TGC AAT GAC ATG ACT CCA GAG CAA ATG GCT ACA
AAT GTG AAC TGT TCC AGC CCT GAG CGA CAC ACA
AGA AGT TAT GAT TAC ATG GAA GGA GGG GAT ATA
AGA GTG AGA AGA CTC TTC TGT CGA ACA CAG TGG
TAC CTG AGG ATC GAT AAA AGA GGC AAA GTA AAA
GGG ACC CAA GAG ATG AAG AAT AAT TAC AAT ATC
ATG GAA ATC AGG ACA GTG GCA GTT GGA ATT GTG
GCA ATC AAA GGG GTG GAA AGT GAA TTC TAT CTT
GCA ATG AAC AAG GAA GGA AAA CTC TAT GCA AAG
AAA GAA TGC AAT GAA GAT TGT AAC TTC AAA GAA
CTA ATT CTG GAA AAC CAT TAC AAC ACA TAT GCA
TCA GCT AAA TGG ACA CAC AAC GGA GGG GAA ATG
TTT GTT GCC TTA AAT CAA AAG GGG ATT CCT GTA
AGA GGA AAA AAA ACG AAG AAA GAA CAA AAA ACA
GCC CAC TTT CTT CCT ATG GCA ATA ACT
TAA(stop)

KGF is a protein of 29 KDa and 194 amino acids.

KGF Protein Sequence:

(SEQ ID NO: 2)
CNDMTPEQMATNVNCSSPERHTRSYDYMEGGDIRVRRL
FCRTQWYLRIDKRGKVKGTQEMKNNYNIMEIRTVAVGI
VAIKGVESEFYLAWINKEGKLYAKKECNEDCNFKELIL
ENHYNTYASAKWTHNGGEMFVALNQKGIPVRGKKTKKE
QKTAHFLPMAIT

The KGF cDNA was amplified by human fibroblasts using Pfu DNA polymerase (Promega) and the following primers: F: 5′-ggatcctgcaatgacatgactccagagc-3′ (SEQ ID NO:4); R: 5′-gagctcttaagttattgccataggaagaaag-3′ (SEQ ID NO:5). The amplification was performed according to the following program: denaturation at 95° C. for 45 seconds, pairing at 59° C. for 30 seconds, extension at 72° C. for 80 seconds; 35 PCR cycles were conducted. The PCR product was visualized by electrophoresis on 1% agarose gel.

Cloning of the KGF Gene in the pET30a Vector

Purification of PCR product from the agarose gel was performed using the Wizard Gel extraction kit and the PCR Clean-up (Promega) system. The purified KGF was cloned in the vector PJet1.2/blunt (Fermentas) by T4 DNA ligase (Fermentas) and the ligated products were transformed into E. coli DH5a cells. Plasmid DNA isolation was performed using the Pure Yield Plasmid Mini Prep (Promega) system as described by the manufacturer. The recombinant plasmids were controlled by digestion with restriction enzymes and DNA sequencing. The plasmid DNA was then digested by the restriction enzymes BAMHI and SacI (New England Biolabs) and transferred into an expression vector pET30a (Novagen, cod. 69909-3) using T4 DNA ligase (Fermentas). The ligated products were transformed into cells of E. coli TOP10 (Invitrogen). The recombinant plasmids were extracted and confirmed by digestion with restriction enzymes and DNA sequencing.

Construction of the Usp45-KGF Plasmid

Usp45 Gene Sequence:

(SEQ ID NO: 6)
ATG AAA AAA AGA TTA TCT CAG CTA TTT TAA TGT
CTA CAG TGA TCC TTA AGT GCT GCA GCC CCG TTG
TCA GGT GTT TAC GCT GAT

Usp45 Protein Sequence:

(SEQ ID NO: 7)
MKKKIISAILMSTVILSAAAPLSGVYAD

A synthetic sequence of Usp45 including restriction enzyme recognition sites was designed by pairing the following primers: F: 5′-atcttcatgaaaaaaaagattatctcagctattttaatgtctacagtgatcttaagtgctgcagccccgttgtcaggtgtttacgc tgatg-3′(SEQ ID NO:8); R: 5′-gatccatcagcgtaaacacctgacaacggggctgcagcacttaagatcactgtagacttaaaatactgagat aatcttttttttcatgaa-3′ (SEQ ID NO:9). The DNA fragments of Usp45 were phosphorylated and bound by T4 DNA ligase (Fermentas) to a pET30a-KGF expression vector digested with the restriction enzymes BamHI and BglII and dephosphorylated. In this way a recombinant plasmid was obtained in which the Usp45 sequence is joined to the KGF coding sequence by a 6 nucleotide linker containing a cutting site for the restriction enzyme BamHI. The ligated products were transformed into E. coli TOP10 cells, and the recombinant plasmids were confirmed by digestion with appropriate restriction enzymes. Due to the presence of a cutting site of the BamHI restriction enzyme between the sequence of Usp45 and KGF, the final protein encoded by the pET30a-Usp45-KGF expression plasmid differed from the original KGF sequence for to the presence of 3 more amino acids. For this reason, the correct KGF protein sequence was obtained by substitution with an in vitro synthesized sequence in which the nucleotides coding for the three additional amino acids were removed. The Usp45-KGF insert was first transferred to the pGL3-basic vector [Promega, cat. N. E1751], and therefore the new synthetic sequence was inserted in the correct position through the digestion with restriction enzymes Bpu10I and PstI, followed by ligation. The insert was then confirmed by DNA sequencing.

Construction of the Recombinant L. lactis Strain NZ3900/pNZ8149-Usp45-KGF for Food Use

A strain of L. lactis capable of secreting KGF was generated by transformation of the strain NZ3900 of L. lactis (MoBiTec-Molecular Biotechnology), for food use, with an expression plasmid containing KGF fused to the Usp45 secretion signal. The L. lactis strains were grown at 30° C. in liquid M17 medium supplemented with lactose or glucose at 0.5% and 10 μg/ml of chloramphenicol or erythromycin. The NZ3900 strain is a standard strain for food selection based on the ability to grow on lactose. This strain is a progeny of NZ3000, a strain in which the lactose operon, which is generally present on the plasmids, has been integrated into the chromosome, and the lacF gene has been eliminated. The lacF gene deletion renders this strain incapable of growing on lactose, unless the lacF is supplied on a plasmid [de Ruyter et al., 1996]. Thus, the host strain of L. lactis NZ3900 can grow on glucose, but in the presence of the lacF gene on a plasmid, it can also grow on lactose. Therefore, the commercial vector for L. lactis pNZ8149 (MoBiTec) [Mierau et al., 2005], which contains lacF as a food selection marker, was chosen for the expression of KGF so that the transformed cells can be selected for the ability to grow on lactose. The Elliker medium (tryptone, yeast extract, sodium chloride, sodium acetate, ascorbic acid, agar) was used for the selection of the Lac+colonies. On this rich medium both Lac+ and Lac-cells can grow, but when lactose is added as the sole source of carbon, the lactose fermenting cells provide yellow colonies. To generate the plasmid pNZ8149-Usp45-KGF (for brevity indicated from now on as PNZKGF), the plasmids pNZ8149 from L. lactis and pGL3 basic-Usp45-KGF were digested with the restriction enzymes NcoI and SacI and then ligated. The resulting plasmid was transferred into competent cells of L. lactis by electroporation. In short, the competent NZ3900 cells of L. lactis were prepared by two successive sub-inoculations in M17 medium containing 0.5% glucose (GM17 medium). Then 5 mL of culture medium were transferred to 50 ml of GM17 medium and incubated for about 3 hours at 30° C., until an OD600 nm of 0.3 was reached. The cells were collected by centrifugation (6000 rpm, 20 min, 4° C.), subsequently washed in 400, 200, 100 and then 40 mL of sterile buffer (0.5 M sucrose, 10% glycerol) and centrifuged again (6000 rpm, 20 min, 4° C.). The cells were finally resuspended in 4 mL of sterile buffer and stored at 4° C. Then, 40 μL of cells were placed in a pre-chilled electroporation cuvette with 1 μL of DNA (pNZKGF, in TE buffer) which was kept on ice during electroporation (2000 V, 25 μF, 200Ω and a pulse from 4.5 to 5 ms). After electroporation, 1 ml of GM17 medium, CaCl2 (2 mM) and MgCl₂ (20 mM) were added to the cuvette, then incubated on ice for 5 minutes and then at 30° C. for 1.5 hours. After electroporation, the bacteria were plated on Elliker agar (for the selection of Lac+transformants) and incubated for 1 or 2 days at 30° C.

Induction of KGF Protein Expression and Secretion

KGF expression was induced by the addition of sub-inhibitory amounts of nisin to the culture medium. Briefly, 5 ml of L. lactis NZ3900 cells transformed with the pNZKGF plasmid were grown overnight at 30° C., then diluted 1/25 in 2×10 ml of fresh medium and grown at 30° C. until reaching an OD600 nm equal to 0.4. 10 ml of culture were induced with 1 ng/ml of nisin, while the other 10 ml of culture, not induced, were used as a negative control. The cells were incubated for 3 hours and the OD600 nm was measured to monitor the growth of induced and non-induced cultures. The cells were collected by centrifugation at 6.000 rpm for 5 minutes. Both cell lysates and supernatants were obtained and tested for the presence of the KGF protein.

KGF Secretion Optimization

The optimization of the KGF secretion was performed by modifying various parameters, such as the collection time after Nisin induction and the Nisin concentration, to find the optimal conditions for the expression and secretion of the recombinant protein. Different concentrations of Nisin (0.5, 0.75 and 1 ng/ml) and different collection times after induction (3, 12 and 24 hours) were examined.

KGF Protein Expression

The cell lysates of L. lactis, induced or not with 0.5 ng/ml of Nisin for 3, 12 or 24 hours, were analyzed at the protein level by SDS-PAGE. 15 μL of each sample were loaded onto 15% polyacrylamide gel. The proteins were visualized by staining with blue coomassie.

ELISA Assay

The supernatants of L. lactis cells were collected, induced or not with 0.5, 0.75 or 1 ng/ml of Ni sin for 3, 12 or 24 hours. The concentration of KGF in the induced and non-induced L. lactis supernatants was measured using a standard ELISA kit (R&D Systems), as recommended by the manufacturer. The sensitivity of the ELISA test is ≥10 pg/mL. The values are presented in graph and table as mean±standard deviation.

Western Blot Analysis

Primary cultures of cells of the human vaginal mucosa (HVM) have been established starting from 1 cm² of full-thickness biopsy of the vaginal mucosa, as previously reported [Panici et al., 2007] [informed consent from patients was obtained], and maintained in basal medium for keratinocytes added with growth factor aliquots (KGM, Lonza), with change of medium twice a week. The cells were treated for 30 minutes with recombinant human KGF (Upstate Biotechnology), as a positive control, or with Nisin-induced L. lactis supernatants for 12 or 24 hours. The cells were lysed in the RIPA buffer. Total proteins (50-150 ng) were run under reducing conditions by SDS-PAGE on 10% polyacrylamide gel and transferred to Immobilon-FL membranes (Merck Millipore). The membranes were blocked in TBS buffer containing 0.1% Tween 20 (TBS-T) and 5% milk for 1 hour at 25° C. and then incubated overnight at 4° C. with the following primary antibodies: anti-phospho-p44/42 MAPK (Thr202/Tyr204) (Cell Signaling Technology) and anti-ERK2 (Santa Cruz Biotechnology). The membranes were then incubated with secondary antibody conjugated with horseradish peroxidase (HRP) (Sigma-Aldrich) for 1 hour at 25° C. The bound antibody was detected by enhanced chemiluminescent detection reagents (Pierce Biotechnology Inc), according to the manufacturer's instructions. Densitometric analysis was performed with the Quantity One program (Bio-Rad Laboratories).

Results

KGF Gene Amplification and Cloning

The KGF gene was amplified from human fibroblasts by PCR using appropriate primers, and visualized on a 1% agarose gel, in which a 500-bp product is shown, compatible with the KGF gene dimensions (FIG. 1). Thus, the KGF gene was cloned into an intermediate vector (pJet1.2/blunt, FIG. 2A). HindIII and EcoRI restriction enzymes were used to digest the plasmids extracted from positive colonies, and the presence of the expected 3000 and 500 bp fragments confirmed the correct insertion of the KGF gene in the vector pJet1.2/blunt (FIG. 2B). Then, the KGF insert was transferred to the pET30a vector (FIG. 3A). SphI and XhoI restriction enzymes were used to digest the plasmids extracted from positive colonies, and the presence of the expected fragments, of 990 and 4700-bp, confirmed the correct insertion of the KGF gene in the pET30a vector (FIG. 3B).

Construction of the Usp45-KGF Plasmid

In order to obtain the extracellular secretion of KGF in L. lactis cells, it was necessary to insert a specific secretion signal peptide for L. lactis upstream of the KGF sequence. The signal peptides mainly used for protein secretion in L. lactis are:

(1) the signal peptide of the main secreted lactococcal proteins, Usp45 and

(2) the proteinase signal peptide associated with the PrtP cell wall [sequence aa: MQRKKKGLSFLLAGTVALGALAVLPVGEIQAKA (SEQ ID NO: 10)]. Generally, the signal peptide of Usp45 provides better results and is more widespread than that of PrtP. Thus, inventors have cloned the synthetic gene sequence of Usp45 upstream of the KGF gene in the intermediate vector pGL3-basic (FIG. 4A). The restriction enzymes HindIII and BglII were used to digest the plasmids extracted from positive colonies, and the presence of the expected Usp45-KGF fragment (590 bp) confirmed the insertion of the Usp45-KGF construct in the pGL3-basic vector (FIG. 4B).

Construction of Recombinant L. lactis Food Use Able to Inducibly Secrete KGF

The goal was to obtain a L. lactis strain able to secrete KGF inducibly, in order to obtain a prolonged release of KGF at the level of the vaginal mucosa. To this end, the Nisin-controlled gene expression system (NICE), developed by NIZO Food Research, was used. Briefly, the Usp45-KGF insert was extracted from pGL3-basic by digestion with the restriction enzymes NcoI and SacI, and cloned in a pNZ8149 vector digested with the same enzymes (FIG. 5A). In the plasmid pNZ8149, the gene of interest is under the control of the inducible promoter PnisA, so its expression can be induced by the addition of sub-inhibitory amounts of nisin (0.1-5 ng/ml) to the culture medium. The pNZKGF plasmid thus obtained was then transferred by electroporation into the L. lactis NZ3900 strain, specifically developed for food applications of the NICE system (FIG. 5B). Such strain is derived from the NZ3000 strain, in which the lactose operon, which is generally present on the plasmids, has been integrated into the chromosome, and the lacF gene has been eliminated. The lacF gene deletion renders this strain incapable of growing on lactose, unless lacF is supplied on a plasmid, such as pNZ8149 [de Ruyter et al., 1996]. Thus, the positive transformants were selected based on their ability to grow on lactose.

KGF Protein Expression and Secretion

The L. lactis NZ3900 cells transformed with the pNZKGF plasmid were induced or not with 0.5 ng/ml of nisin for 3, 12 or 24 hours, and then collected by centrifugation. KGF protein expression was studied by SDS-PAGE and subsequent Comassie blue staining. In the induced cells a band of 18 kDa was identified, corresponding to the molecular weight of the KGF (FIG. 6, arrow). The results of SDS PAGE showed that by inducing with 0.5 ng/ml of Nisin the maximum concentration of KGF was obtained 24 hours after induction. The KGF protein produced by L. lactis should be secreted within the medium due to the presence of the Usp45 secretion signal. Thus, KGF secretion at different doses of Nisin and at various induction times was analyzed by ELISA on L. lactis cell supernatants, as shown in FIG. 7. The highest amount of KGF in the culture medium was obtained by inducing with 1 ng/ml of Nisin for 24 hours.

Effectiveness of KGF Secreted by L. lactis

The functionality of KGF secreted by the induced L. lactis cells was evaluated by analysing its ability to induce activation of the ERK pathway in vaginal mucosa cells (HVM). Inventors therefore analyzed ERK phosphorylation in HVM cells after treatment with L. lactis supernatants at 12 or 24 hours of induction with 1 ng/ml of Nisin. Non-induced cell supernatants were used as a negative control, while human recombinant KGF was used as a positive control. Using Western blot, we showed an activation of MAPK ERK1 and 2 after 30 minutes of treatment with both induced supernatants, comparable to that observed after treatment with KGF (FIG. 8A), as also documented by densitometric analysis (FIG. 8B).

### CONCLUSIONS

The present authors have engineered a lactobacillus compatible with the vaginal microenvironment (L. lactis) to produce KGF inducibly. The procedure used is shown in FIG. 9. First, the KGF gene was amplified by human fibroblasts. To obtain the release of KGF from the L. lactis cells, a construct was generated in which the secretion signal peptide Usp45, specific for L. lactis, was inserted upstream of the sequence coding for the KGF (1). This construct was cloned into intermediate vectors (2), and then into the final vector, pNZ8149 (3). To obtain a controlled production of KGF, the pNZKGF vector was transferred by electroporation into a strain of L. lactis (NZ3900) bearing a NICE (4) expression system. Induction with nisin was then performed to allow the expression and secretion of the KGF protein (5), which were evaluated by SDS-PAGE and by ELISA assay, respectively (6). Finally, the effectiveness of KGF produced by L. lactis was tested by Western blot analysis in human vaginal cells (7).

### REFERENCES

Barasch A, Epstein J, Tilashalski K. Palifermin for management of treatment-induced oral mucositis in cancer patients. Biologics. 2009; 3:111-6.
Beaven A W, Shea T C.
The effect of palifermin on chemotherapy and radiation therapy-induced mucositis: a review of the current literature. Support Cancer Ther. 2007 Sep. 1; 4(4):188-97
Bermúdez-Humarán L G, Corthier G, Langella P. Recent advances in the use of Lactococcus lactis as live recombinant vector for the development of new safe mucosal vaccines. Recent Res Devel Microbiology 2004; 8:147-60.
Bermúdez-Humarán LG. Lactococcus lactis as a live vector for mucosal delivery of therapeutic proteins. Hum Vaccin. 2009 April; 5(4):264-7.
Bolotin A, Mauger S, Malarme K, Ehrlich S D, Sorokin A. Low-redundancy sequencing of the entire Lactococcus lactis IL1403 genome. Antonie Van Leeuwenhoek. 1999 July-November; 76(1-4):27-76.
Brauchle M, Madlener M, Wagner A D, Angermeyer K, Lauer U, Hofschneider P H, Gregor M, Werner S. Keratinocyte growth factor is highly overexpressed in inflammatory bowel disease. Am J Pathol. 1996 August; 149(2):521-9.
Brizel D M, Murphy B A, Rosenthal D I, Pandya K J, Glück S, Brizel H E, Meredith R F, Berger D, Chen M G, Mendenhall W. Phase II study of palifermin and concurrent chemoradiation in head and neck squamous cell carcinoma. J Clin Oncol. 2008 May 20; 26(15):2489-96.
Bron P A, Kleerebezem M. Lactic Acid Bacteria for Delivery of Endogenous or Engineered Therapeutic Molecules. Front Microbiol. 2018 Aug. 3; 9:1821.
Ceccarelli S, D'Amici S, Vescarelli E, Coluccio P, Matricardi P, di Gioia C, Benedetti Panici P, Romano F, Frati L, Angeloni A, Marchese C. Topical KGF treatment as a therapeutic strategy for vaginal atrophy in a model of ovariectomized mice. J Cell Mol Med. 2014 September; 18(9): 1895-907.
Davis S S. Nasal vaccines. Adv Drug Deliv Rev 2001; 51:21-42.
del Carmen S, de Moreno de LeBlanc A, Perdigon G, Bastos Pereira V, Miyoshi A, Azevedo V, LeBlanc J G. Evaluation of the anti-inflammatory effect of milk fermented by a strain of IL-10-producing Lactococcus lactis using a murine model of Crohn's disease. J Mol Microbiol Biotechnol. 2011; 21(3-4): 138-46.
Delves-Broughton J, Blackburn P, Evans R J, Hugenholtz J. Applications of the bacteriocin, nisin. Antonie Van Leeuwenhoek. 1996 February; 69(2):193-202.
de Ruyter P G, Kuipers O P, de Vos W M. Controlled gene expression systems for Lactococcus lactis with the food-grade inducer nisin. Appl Environ Microbiol. 1996 October; 62(10):3662-7.
de Vos W M. Gene expression systems for lactic acid bacteria. Curr Opin Microbiol. 1999 June; 2(3):289-95.
Edey K A, Rundle S, Hickey M. Hormone replacement therapy for women previously treated for endometrial cancer. Cochrane Database Syst Rev. 2018 May 15; 5:CD008830.
Eichenbaum Z, Federle M J, Marra D, de Vos W M, Kuipers O P, Kleerebezem M, Scott J R. Use of the lactococcal nisA promoter to regulate gene expression in gram-positive bacteria: comparison of induction level and promoter strength. Appl Environ Microbiol. 1998 August; 64(8):2763-9.
Eswarakumar V P, Lax I, Schlessinger J. Cellular signaling by fibroblast growth factor receptors. Cytokine Growth Factor Rev. 2005 April; 16(2):139-49.
Finch P W, Rubin J S. Keratinocyte growth factor/fibroblast growth factor 7, a homeostatic factor with therapeutic potential for epithelial protection and repair. Adv Cancer Res. 2004; 91:69-136.
Fuller R. Probiotics in man and animals. J Appl Bacteriol. 1989 May; 66(5):365-78.
Gomperts B N, Belperio J A, Fishbein M C, Keane M P, Burdick M D, Strieter R M. Keratinocyte growth factor improves repair in the injured tracheal epithelium. Am J Respir Cell Mol Biol. 2007 July; 37(1):48-56.
Hoesl C E, Altwein J E. The probiotic approach: an alternative treatment option in urology. Eur Urol. 2005 March; 47(3):288-96.
Holmgren J, Czerkinsky C. Mucosal immunity and vaccines. Nat Med 2005; 11:45-53.
Keys H M, Roberts J A, Brunetto V L, Zaino R J, Spirtos N M, Bloss J D, Pearlman A, Maiman M A, Bell J G; Gynecologic Oncology Group. A phase III trial of surgery with or without adjunctive external pelvic radiation therapy in intermediate risk endometrial adenocarcinoma: a Gynecologic Oncology Group study. Gynecol Oncol. 2004 March; 92(3):744-51.
Kim S, Ko Y, Lee H J, Lim J E. Menopausal hormone therapy and the risk of breast cancer by histological type and race: a meta-analysis of randomized controlled trials and cohort studies. Breast Cancer Res Treat. 2018 August; 170(3):667-675.
Marchese C, Chedid M, Dirsch O R, Csaky K G, Santanelli F, Latini C, LaRochelle W J, Torrisi M R, Aaronson S A. Modulation of keratinocyte growth factor and its receptor in reepithelializing human skin. J Exp Med. 1995 Nov. 1; 182(5):1369-76.
Neutra M R, Kozlowski P A. Mucosal vaccines: the promise and the challenge. Nat Rev Immunol 2006; 6:148-58.
Masui F, Matsuda M, Mori T. Involvement of keratinocyte growth factor (KGF)-KGF receptor signaling in developmental estrogenization syndrome of mouse vagina. Cell Tissue Res. 2004 December; 318(3):591-8.
Pandit L, Ouslander J G. Postmenopausal vaginal atrophy and atrophic vaginitis. Am J Med Sci. 1997; 314 (4):228-231.
Panici P B, Bellati F, Boni T, Francescangeli F, Frati L, Marchese C. Vaginoplasty using autologous in vitro cultured vaginal tissue in a patient with Mayer-von-Rokitansky-Kuster-Hauser syndrome. Hum Reprod. 2007 July; 22(7):2025-8.
Pavan S, Hols P, Delcour J, Geoffroy M C, Grangette C, Kleerebezem M, Mercenier A. Adaptation of the nisin-controlled expression system in Lactobacillus plantarum: a tool to study in vivo biological effects. Appl Environ Microbiol. 2000 October; 66(10):4427-32.
Poelvoorde N, Huyghebaert N, Vervaet C, Remon J P. Optimisation of an enteric coated, layered multi-particulate formulation for ileal delivery of viable recombinant Lactococcus lactis. Eur J Pharm Biopharm. 2008 August; 69(3):969-76.
Pontes D S, de Azevedo M S, Chatel J M, Langella P, Azevedo V, Miyoshi A. Lactococcus lactis as a live vector: heterologous protein production and DNA delivery systems. Protein Expr Purif. 2011 October; 79(2):165-75.
Reid G, Bocking A. The potential for probiotics to prevent bacterial vaginosis and preterm labor. Am J Obstet Gynecol. 2003 October; 189(4):1202-8.
Spielberger R, Stiff P, Bensinger W, Gentile T, Weisdorf D, Kewalramani T, Shea T, Yanovich S, Hansen K, Noga S, McCarty J, LeMaistre C F, Sung E C, Blazar B R, Elhardt D, Chen M G, Emmanouilides C. Palifermin for oral mucositis after intensive therapy for hematologic cancers. N Engl J Med. 2004 December 16; 351(25):2590-8.
Todorov S D, Botes M, Danova S T, Dicks L M. Probiotic properties of Lactococcus lactis ssp. lactis HV219, isolated from human vaginal secretions. J Appl Microbiol. 2007 September; 103(3):629-39.
Werner S, Smola H, Liao X, Longaker M T, Krieg T, Hofschneider P H, Williams L T. The function of KGF in morphogenesis of epithelium and reepithelialization of wounds. Science. 1994 Nov. 4; 266(5186):819-22.
Werner S. Keratinocyte growth factor: a unique player in epithelial repair processes. Cytokine Growth Factor Rev. 1998 June; 9(2): 153-65.

1. A microorganism characterized in that it is genetically modified to express the human growth factor of keratinocytes (KGF/FGF7) or its functional orthologues, derivatives or fragments.

2. The microorganism according to claim 1, wherein the microorganism is a probiotic.

3. The microorganism according to claim 1 being a GRAS organism.

4. The microorganism according to claim 1, that is a lactic acid bacterium.

5. The microorganism according to claim 4, that belongs to the genus Lactococcus.

6. The microorganism according to claim 5, wherein the microorganism that belongs to the genus Lactococcus is a Lactococcus Lactis of the NZ3900 strain.

7. The microorganism according claim 1, wherein the microorganism has been genetically modified with a recombinant polynucleotide comprising a nucleic acid encoding the human KGF, its functional orthologues, derivatives or functional fragments and/or said microorganism comprises a plasmid comprising a nucleic acid encoding the human keratinocyte growth factor (KGF/FGF7) or its functional orthologues, derivatives or fragments.

8. The microorganism according to claim 7, wherein the nucleic acid encoding the human KGF, its functional orthologues, derivatives or fragments is operatively linked to an expression promoter.

9. The microorganism according to claim 8, wherein the promoter is inducible.

10. The microorganism according claim 1, wherein the KGF, its functional orthologues, derivatives or fragments are secreted.

11. The microorganism according claim 1, wherein the KGF, its orthologues, derivatives or functional fragments are expressed as a fusion protein with a secretion signal that works in the microorganism.

12. The microorganism according to claim 1, wherein the nucleic acid encoding the KGF comprises a sequence having at least 80% identity with the SEQ ID NO: 1 or 11.

13. A composition comprising the microorganism according to claim 1, and at least one excipient.

14. A pharmaceutical composition comprising the microorganism according to claim 1, and at least one pharmaceutically acceptable excipient.

15. A combination which comprises:
a) the microorganism according to claim 1, or the microorganism and at least one pharmaceutically acceptable excipient; and
b) an inducer of the expression promoter.

16. A method of treating vaginal atrophy, dysuria, vaginal pain and/or vaginal drying induced by a post-menopausal status, by surgery, by a pathology and/or by chemotherapy or radiotherapy in a subject in need thereof, comprising administering the composition of claim 13.

17. A method of producing human KGF or its functional orthologues, derivatives or fragments directly in situ in a host, by administering the microorganism of claim 1 to the host in need thereof.

18. A method of treating a subject in need thereof by administering the composition according to claim 13, topically on the vagina mucosa,
wherein the composition comprises a microorganism that has been genetically modified with a recombinant polynucleotide comprising a nucleic acid encoding human KGF, its functional orthologues, derivatives or functional fragments and/or said microorganism comprises a plasmid comprising a nucleic acid coding for the human keratinocyte growth factor (KGF/FGF7) or its functional orthologues, derivatives or fragments, and
wherein the nucleic acid encoding the human KGF, its functional orthologues, derivatives or fragments is operatively linked to an inducible expression promoter.

19. The method according to claim 18, wherein an inducer of the expression promoter is also administered.

20. The method according to claim 19, wherein the expression promoter inducer is administered orally.


SEQUENCE LISTING (12 sequences)

SEQ ID NO: 1 - DNA, 492 bp, Homo sapiens
tgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaa

SEQ ID NO: 2 - protein, 163 aa, Homo sapiens
CNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 3 - protein, 194 aa, Homo sapiens
MHKWILTWILPTLLYRSCFHIICLVGTISLACNDMTPEQMATNVNCSSPERHTRSYDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT

SEQ ID NO: 4 - DNA, 28 bp, Artificial Sequence
  PRIMER
ggatcctgcaatgacatgactccagagc

SEQ ID NO: 5 - DNA, 31 bp, Artificial Sequence
  PRIMER
gagctcttaagttattgccataggaagaaag

SEQ ID NO: 6 - DNA, 84 bp, Artificial Sequence
  USP45
atgaaaaaaagattatctcagctattttaatgtctacagtgatccttaagtgctgcagccccgttgtcaggtgtttacgctgat

SEQ ID NO: 7 - protein, 28 aa, Artificial Sequence
  USP45
MKKKIISAILMSTVILSAAAPLSGVYAD

SEQ ID NO: 8 - DNA, 91 bp, Artificial Sequence
  PRIMER
atcttcatgaaaaaaaagattatctcagctattttaatgtctacagtgatcttaagtgctgcagccccgttgtcaggtgtttacgctgatg

SEQ ID NO: 9 - DNA, 90 bp, Artificial Sequence
  PRIMER
gatccatcagcgtaaacacctgacaacggggctgcagcacttaagatcactgtagacttaaaatactgagataatcttttttttcatgaa

SEQ ID NO: 10 - protein, 33 aa, Artificial Sequence
  PrtP
MQRKKKGLSFLLAGTVALGALAVLPVGEIQAKA

SEQ ID NO: 11 - DNA, 3936 bp, Homo sapiens
agttttaattgcttccaatgaggtcagcaaaggtatttatcgaaaagccctgaataaaaggctcacacacacacacaagcacacacgcgctcacacacagagagaaaatccttctgcctgttgatttatggaaacaattatgattctgctggagaacttttcagctgagaaatagtttgtagctacagtagaaaggctcaagttgcaccaggcagacaacagacatggaattcttatatatccagctgttagcaacaaaacaaaagtcaaatagcaaacagcgtcacagcaactgaacttactacgaactgtttttatgaggatttatcaacagagttatttaaggaggaatcctgtgttgttatcaggaactaaaaggataaggctaacaatttggaaagagcaactactctttcttaaatcaatctacaattcacagataggaagaggtcaatgacctaggagtaacaatcaactcaagattcattttcattatgttattcatgaacacccggagcactacactataatgcacaaatggatactgacatggatcctgccaactttgctctacagatcatgctttcacattatctgtctagtgggtactatatctttagcttgcaatgacatgactccagagcaaatggctacaaatgtgaactgttccagccctgagcgacacacaagaagttatgattacatggaaggaggggatataagagtgagaagactcttctgtcgaacacagtggtacctgaggatcgataaaagaggcaaagtaaaagggacccaagagatgaagaataattacaatatcatggaaatcaggacagtggcagttggaattgtggcaatcaaaggggtggaaagtgaattctatcttgcaatgaacaaggaaggaaaactctatgcaaagaaagaatgcaatgaagattgtaacttcaaagaactaattctggaaaaccattacaacacatatgcatcagctaaatggacacacaacggaggggaaatgtttgttgccttaaatcaaaaggggattcctgtaagaggaaaaaaaacgaagaaagaacaaaaaacagcccactttcttcctatggcaataacttaattgcatatggtatataaagaaccagttccagcagggagatttctttaagtggactgttttctttcttctcaaaattttctttccttttattttttagtaatcaagaaaggctggaaaactactgaaaaactgatcaagctggacttgtgcatttatgtttgttttaagacactgcattaaagaaagatttgaaaagtatacacaaaaatcagatttagtaactaaaggttgtaaaaaattgtaaaactggttgtacaatcatgatgttagtaacagtaatttttttcttaaattaatttacccttaagagtatgttagatttgattatctgataatgattatttaaatattcctatctgcttataaaatggctgctataataataataatacagatgttgttatataaggtatatcagacctacaggcttctggcaggatttgtcagataatcaagccacactaactatggaaaatgagcagcattttaaatgctttctagtgaaaaattataatctacttaaactctaatcagaaaaaaaattctcaaaaaaactattatgaaagtcaataaaatagataatttaacaaaagtacaggattagaacatgcttatacctataaataagaacaaaatttctaatgctgctcaagtggaaagggtattgctaaaaggatgtttccaaaaatcttgtatataagatagcaacagtgattgatgataatactgtacttcatcttacttgccacaaaataacattttataaatcctcaaagtaaaattgagaaatctttaagtttttttcaagtaacataatctatctttgtataattcatatttgggaatatggcttttaataatgttcttcccacaaataatcatgcttttttcctatggttacagcattaaactctattttaagttgtttttgaactttattgttttgttatttaagtttatgttatttataaaaaaaaaaccttaataagctgtatctgtttcatatgcttttaattttaaaggaataacaaaactgtctggctcaacggcaagtttccctcccttttctgactgacactaagtctagcacacagcacttgggccagcaaatcctggaaggcagacaaaaataagagcctgaagcaatgcttacaatagatgtctcacacagaacaatacaaatatgtaaaaaatctttcaccacatattcttgccaattaattggatcatataagtaaaatcattacaaatataagtatttacaggattttaaagttagaatatatttgaatgcatgggtagaaaatatcatattttaaaactatgtatatttaaatttagtaattttctaatctctagaaatctctgctgttcaaaaggtggcagcactgaaagttgttttcctgttagatggcaagagcacaatgcccaaaatagaagatgcagttaagaataaggggccctgaatgtcatgaaggcttgaggtcagcctacagataacaggattattacaaggatgaatttccacttcaaaagtctttcattggcagatcttggtagcactttatatgttcaccaatgggaggtcaatatttatctaatttaaaaggtatgctaaccactgtggttttaatttcaaaatatttgtcattcaagtccctttacataaatagtatttggtaatacatttatagatgagagttatatgaaaaggctaggtcaacaaaaacaatagattcatttaattttcctgtggttgacctatacgaccaggatgtagaaaactagaaagaactgcccttcctcagatatactcttgggagagagcatgaatggtattctgaactatcacctgattcaaggactttgctagctaggttttgaggtcaggcttcagtaactgtagtcttgtgagcatattgagggcagaggaggacttagtttttcatatgtgtttccttagtgcctagcagactatctgttcataatcagttttcagtgtgaattcactgaatgtttatagacaaaagaaaatacacactaaaactaatcttcattttaaaagggtaaaacatgactatacagaaatttaaatagaaatagtgtatatacatataaaatacaagctatgttaggaccaaatgctctttgtctatggagttatacttccatcaaattacatagcaatgctgaattaggcaaaaccaacatttagtggtaaatccattcctggtagtataagtcacctaaaaaagacttctagaaatatgtactttaattatttgtttttctcctatttttaaatttattatgcaaattttagaaaataaaatttgctctagttacacacctttagaattctagaatattaaaactgtaaggggcctccatccctcttactcatttgtagtctaggaaattgagattttgatacacctaaggtcacgcagctgggtagatatacagctgtcacaagagtctagatcagttagcacatgctttctactcttcgattattagtattattagctaatggtctttggcatgtttttgttttttatttctgttgagatatagcctttacatttgtacacaaatgtgactatgtcttggcaatgcacttcatacacaatgactaatctatactgtgatgatttgactcaaaaggagaaaagaaattatgtagttttcaattctgattcctattcaccttttgtttatgaatggaaagctttgtgcaaaatatacatataagcagagtaagccttttaaaaatgttctttgaaagataaaattaaatacatgagtttctaacaattaga

SEQ ID NO: 12 - protein, 139 aa, Homo sapiens
YDYMEGGDIRVRRLFCRTQWYLRIDKRGKVKGTQEMKNNYNIMEIRTVAVGIVAIKGVESEFYLAMNKEGKLYAKKECNEDCNFKELILENHYNTYASAKWTHNGGEMFVALNQKGIPVRGKKTKKEQKTAHFLPMAIT